(19) United States
(12) Patent Application Publication
(10) Pub. No.: US 2003/0176876 A1
(43) Pub. Date: Sep. 18, 2003

(54) Multi-channel bioresorbable nerve regeneration conduit and process for preparing the same

(76) Inventors: Jui-Hsiang Chen, Hsinchu (TW); Jean-Dean Yang, Dayuan Shiang (TW); Hsin-Hsin Shen, Hsinchu (TW); Yu-Lin Hsieh, Kaohsiung (TW); Bin-Hong Tsai, Kaohsiung (TW); Chiung-Lin Yang, Hsinchu (TW); Mei-Jun Liu, Gungguan Shiang (TW)

Correspondence Address: MERCHANT & GOULD PC, P.O. BOX 2903, MINNEAPOLIS, MN, 55402-0903, US

(21) Appl. No.: 10/161,914

(22) Filed: Jun. 4, 2002

(30) Foreign Application Priority Data
Mar. 11, 2002 (TW) 91104507

(51) Int. Cl. A61B 17/08

(52) U.S. Cl. 606/152

(57) ABSTRACT

A multi-channel bioresorbable nerve regeneration conduit and a process for preparing the conduit. The multi-channel bioresorbable nerve regeneration conduit includes a hollow round tube of a porous bioresorbable polymer and a multi-channel filler in the round tube. The multi-channel filler is a porous bioresorbable polymer film with an uneven surface and is single layer, multiple layer, in a folded form, or wound into a spiral shape.

### BACKGROUND OF THE INVENTION

1. Field of the Invention

The present invention relates to a multi-channel bioresorbable nerve regeneration conduit, and more particularly to a nerve regeneration conduit including a hollow round tube of a porous bioresorbable polymer and a multi-channel filler in the hollow round tube. The multi-channel filler is a porous bioresorbable polymer film with an uneven surface.

2. Background of the Invention:

After biomaterials or devices made of bioresorbable polymers are implanted into a subject for a period of time, the bioresorbable polymers will gradually degrade by hydrolysis or enzymosis. The molecular chain of the original polymer will break down into smaller molecular weight compounds that can be absorbed by biological tissues. This bioresorbable property decreases undesirable foreign body reaction when the polymer material is implanted.

In recent years, using bioresorbable polymer to prepare nerve conduits has drawn many researchers' attention. The nerve conduit obtained can be implanted into a lacerated or severed nerve for repair. Various bioresorbable polymers have been used to prepare nerve conduits, including synthetic and natural polymers. Synthetic bioresorbable polymers include polyglycolic acid (PGA), polylactic acid (PLA), poly(glycolic-co-lactic acid (PLGA), and polycaprolactone (PCL). Natural bioresorbable polymers include collagen, gelatin, silk, chitosan, chitin, alginate, hyaluronic acid, and chondroitin sulphate.

Stensaas et al. in U.S. Pat. Nos. 4,662,884 and 4,778,467 use a non-resorbable material, such as PU, silicone, Teflon®, and nitrocellulose to fabricate a nerve conduit that can inhibit neuroma growth.

Barrows et al. in U.S. Pat. Nos. 4,669,474 and 4,883,618 use a bioresorbable material, such as PLA, PGA, polydioxanone, poly(lactide-co-glycolide), to fabricate a porous tubular device by sintering and bonding techniques. The porous device has a porosity of 25% to 95%.

Griffiths et al. in U.S. Pat. No. 4,863,668 use alternating layers of fibrin and collagen to fabricate a nerve regeneration conduit. A Teflon® coated cylindrical mandrel is dipped in a collagen solution, dried, and dipped in a fibrin solution. The process of dipping is repeated until the desired numbers of layers is reached. Finally, the coated mandrel is placed in a solution of glutaraldehyde/formaldehyde for 30 minutes for cross-linking.

Valentini in U.S. Pat. No. 4,877,029 uses a semi-permeable material, such as acrylic copolymer and polyurethane isocyanate, to fabricate a guidance channel in regenerating nerves.

Yannas et al. in U.S. Pat. No. 4,955,893 disclose a method for producing a biodegradable polymer having a preferentially oriented pore structure by an axial freezing process and a method for using the polymer to regenerate damaged nerve tissue. Preferably, the biodegradable polymer is uncross-linked collagen-glycosaminoglycan.

Li in U.S. Pat. Nos. 4,963,146 and 5,026,381 disclose hollow conduits whose walls are composed of Type I collagen, which has a multi-layered and semi-permeable structure. The pore size of the hollow conduit is 0.006 μm to 5 μm. Nerve growth factors can pass through the pore, but the fibroblasts can not. A precipitating agent such as ammonium hydroxide is added to a Type I collagen dispersion to form a fibrous precipitate. The fibrous precipitate is then contacted with a spinning mandrel to form a conduit, which is then compressed, has supernatant liquid removed, is freeze-dried, and cross-linked with a cross-linking agent such as formaldehyde.

Nichols in U.S. Pat. No. 5,019,087 discloses a hollow conduit composed of a matrix of Type I collagen and laminin-containing material, which is used to promote nerve regeneration across a gap of a severed nerve. The conduit has an inner diameter of 1 mm to 1 cm depending upon the gap size of the severed nerve. The wall of the conduit is 0.05 to 0.2 mm thick.

Mares et al. in U.S. Pat. No. 5,358,475 disclose a nerve channel made from high molecular weight lactic acid polymers, which provides beneficial effect on growth of damaged nerves. However, the lactic acid polymer having a molecular weight of 234,000 to 320,000 does not have obvious effect.

Della Valle et al. in U.S. Pat. No. 5,735,863 disclose biodegradable guide channels for use in nerve treatment and regeneration. A hyaluronic acid ester solution is coated on the surface of a rotating steel mandrel. Next, molten hyaluronic acid ester in fibrous form is wound onto the rotating mandrel. Thus, a tubular bioresorbable device is formed.

Dorigatti et al. in U.S. Pat. No. 5,879,359 disclose a medical device including biodegradable guide channels for use in the repair and regeneration of nerve tissue. The guide channel includes interlaced threads imbedded in a matrix, and both the threads and matrix are made of hyaluronic acid ester.

Hadlock et al. in U.S. Pat. No. 5,925,053 disclose a multi-lumen guidance channel for promoting nerve regeneration and a method for manufacturing the guidance channel. A plurality of wires are placed in a mold. A polymer solution is injected into the mold, solidified by freezing, and dried by sublimation, forming a porous matrix. Finally, the wires are drawn to form a multi-lumen guidance channel with 5 to 5000 lumens. The inner diameter of the lumen is 2 to 500 microns. Schwann cells can be seeded onto the interior surfaces of the lumens.

Aldini et al. in Biomaterials, 1996, Vol. 17, No. 10, pp. 959-962, use a copolymer of L-lactide and ε-caprolactone to prepare a conduit for nerve regeneration. The conduit has an inner diameter of 1.3 mm and a wall thickness of 175 μm.

Kiyotani et al. use polyglycolic acid (PGA) as a starting material to prepare a nerve guide tube with a mesh structure. The tube is coated with collagen and filled with neurotrophic factors such as nerve growth factor, basic fibroblast growth factor and laminin-containing gel (Brain Research, 1996, Vol. 740, pp.66-74).

Den Dunnen et al. use poly(DL-lactide-ε-caprolacton) to prepare a nerve conduit with an inner diameter of 1.5 mm and a wall thickness of 0.30 mm (Journal of Biomedical Materials Research, 1996, Vol. 31, pp. 105-115).

Widmer et al. use a combined solvent casting and extrusion technique to fabricate a porous tubular conduit of two bioresorbable materials, poly(DL-lactic-co-glycolic acid) (PLGA) and poly(L-lactic acid) (PLLA) (Biomaterials, 1998, Vol. 21, pp.1945-1955).

Evans et al. use poly(L-lactic acid) (PLLA) to prepare a porous nerve conduit for repairing sciatic nerve defect in rats. The conduit has an inner diameter of 1.6 mm, an outer diameter of 3.2 mm, and a length of 12 mm (Biomaterials, 1999, Vol. 20, pp. 1109-1115).

Rodriguez et al. compare regeneration effect after sciatic nerve resection and tubulization repair with 8 mm bioresorbable guides of poly(L-lactide-co-ε-caprolactone) (PLC) and permanent guides of polysulfone (POS) with different degrees of permeability, leaving a 6 mm gap in different groups of mice (Biomaterials, 1999, Vol. 20, pp. 1489-1500).

Suzuki et al. use alginate gel to prepare a bioresorbable artificial nerve guide by freeze-drying and evaluate its effect on peripheral nerve regeneration using a cat sciatic nerve model (Neuroscience Letters, 1999, Vol. 259, pp. 75-78).

In Steuer et al., polylactide fibers are treated with oxygen plasma, coated with poly-D-lysine, and adhered with Schwann cells (Neuroscience Letters, 1999, Vol. 277, pp. 165-168).

Matsumoto et al. use polyglycolic acid (PGA) and collagen to prepare an artificial nerve conduit. Laminin-coated collagen fibers are then filled in the conduit (Brain Research, 2000, Vol. 868, pp. 315-328).

Wan et al. disclose a method for fabricating polymeric conduits from P(BHET-EOP/TC) and a method on how to control porosity (Biomaterials, 2001, Vol. 22, pp. 1147-1156).

Wang et al. use poly(phosphoester) (PPE) to fabricate two nerve guide conduits with different molecular weight and different polydispersity (PI) (Biomaterials, 2001, Vol. 22, pp. 1157-1169).

Meek et al. use poly(DLLA-ε-CL) to fabricate a thin-walled nerve guide. Modified denatured muscle tissue (MDMT) is filled in the nerve guide in order to support the guide structure and prevent collapse (Biomaterials, 2001, Vol. 22, pp. 1177-1185).

### SUMMARY OF THE INVENTION

The object of the present invention is to provide a multi-channel bioresorbable nerve regeneration conduit.

Another object of the present invention is to provide a process for preparing a multi-channel bioresorbable nerve regeneration conduit.

To achieve the above-mentioned objects, the multi-channel bioresorbable nerve regeneration conduit of the present invention includes a hollow round tube of a porous bioresorbable polymer; and a multi-channel filler in the round tube. The multi-channel filler is a porous bioresorbable polymer film with an uneven surface and is single layer, multiple layer, in a folded form, or wound into a spiral shape.

The process for preparing a porous bioresorbable material having interconnected pores according to the present invention includes the following steps. First, a multi-channel filler is formed, which is a porous bioresorbable polymer film with an uneven surface and is single layer, multiple layer, in a folded form, or wound into a spiral shape. Then, a hollow round tube of a porous bioresorbable polymer is formed. Finally, the multi-channel filler is placed into the hollow round tube.

### BRIEF DESCRIPTION OF THE DRAWINGS

The present invention will become more fully understood from the detailed description given hereinbelow and the accompanying drawings, given by way of illustration only and thus not intended to be limitative of the present invention.

FIGS. 1A to 1F are SEM photographs of the porous PCL film pre-forms obtained from Example (A1) of the present invention, wherein magnification is 350×, 2000×, 100×, 350×, 500×, and 350× respectively.

FIG. 2 is a SEM photograph of the porous PCL film pre-form obtained from Example (A2) of the present invention with magnification of 100×.

FIG. 3 is a SEM photograph of the porous PCL film pre-form obtained from Example (A3) of the present invention with magnification of 3500×.

FIGS. 4A and 4B are SEM photographs of the porous PCL film pre-forms obtained from Example (A4) of the present invention, wherein magnification is 500× and 350× respectively.

FIGS. 5A and 5B are SEM photographs of the porous PCL hollow round tubes obtained from Example (B1) of the present invention, wherein magnification is 200× and 750× respectively.

FIG. 6 is a SEM photograph of the porous PCL hollow round tube obtained from Example (B2) of the present invention with magnification of 200×.

FIG. 7 is a SEM photograph of the porous PCL hollow round tube obtained from Example (B3) of the present invention with magnification of 50×.

FIGS. 8A and 8B are SEM photographs of the multi-channel bioresorbable nerve regeneration conduits obtained from Example (C1) with magnifications of 50× and 35× respectively.

### DETAILED DESCRIPTION OF THE INVENTION

According to a preferred embodiment of the present invention, the structure and preparation of the multi-channel bioresorbable nerve regeneration conduit are described below.

Formation of Multi-Channel Filler of a Porous Bioresorbable Polymer:

First, a bioresorbable polymer is dissolved in an organic solvent to form a bioresorbable polymer solution. Then, the bioresorbable polymer solution is made to have a film shape with an uneven surface. For example, the bioresorbable polymer solution can be coated onto the surface of a mold with an uneven surface or poured into a container.

Subsequently, the film-shaped solution is contacted with a coagulant to form a porous bioresorbable film pre-form having an uneven surface. The bioresorbable polymer solution preferably contacts the coagulant at a temperature of 5° C. to 60° C., and more preferably at a temperature of 10° C. to 50° C. The shape of the film pre-form is not limited, unless at least one surface of the film pre-form is uneven. For example, the porous bioresorbable polymer film with an uneven surface can include a base and a plurality of protrusions protruding from the surface of the base. Preferably, the base has a thickness of 0.05 mm to 1.0 mm, and the protrusion has a protruding depth of 0.05 mm to 1.0 mm.

The bioresorbable film with uneven shape can be a single layer, multiple layer, in a folded form, or wound into a spiral shape, forming a multi-channel filler.

Formation of Hollow Round Tube of a Porous Bioresorbable Polymer:

A bioresorbable polymer is dissolved in an organic solvent to form a bioresorbable polymer solution. Then, the bioresorbable polymer solution is made to have a hollow round tube shape. Then, the hollow round tube-shaped solution is contacted with a coagulant to form a porous bioresorbable hollow round tube.

For example, the bioresorbable polymer solution can be coated onto the surface of a rod to make the solution have a hollow round tube shape. Next, the rod coated with the bioresorbable polymer solution is placed in a coagulant. Thus, a round tube shaped-porous bioresorbable material is formed on the surface of the rod. Finally, the round tube-shaped porous bioresorbable material is drawn out from the surface of the rod, obtaining a porous bioresorbable hollow round tube. The wall thickness of the hollow round tube can be 0.05 to 1.5 mm.

Formation of Multi-Channel Bioresorbable Nerve Regeneration Conduit:

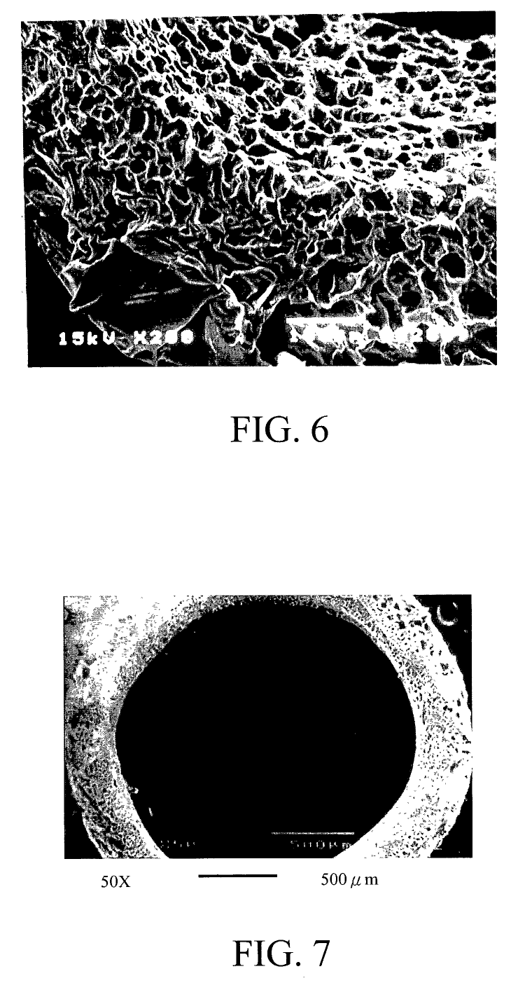

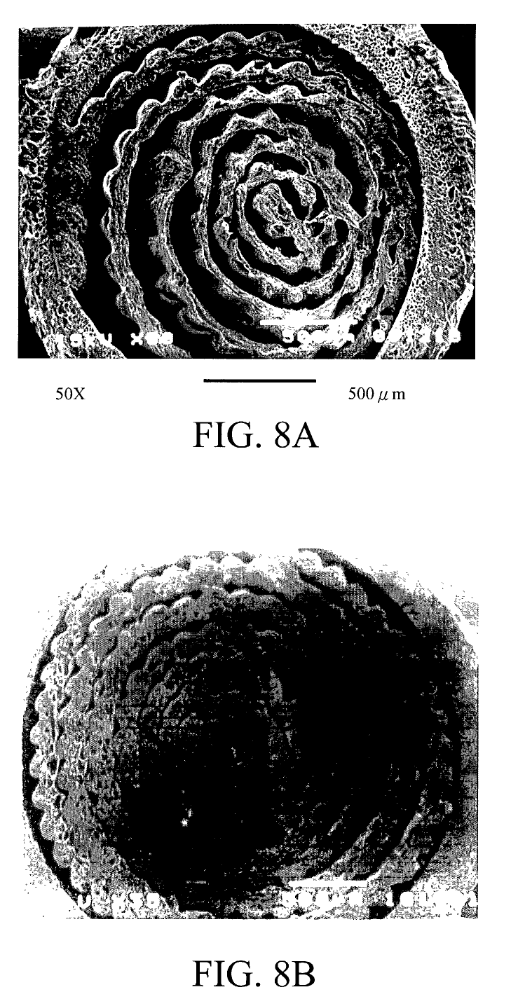

The porous bioresorbable polymer film with uneven surface, which is a single layer, multiple layer, in a folded form, or wound into a spiral shape, is placed into the hollow round tube of a porous bioresorbable polymer (for example, shown in FIG. 7). FIGS. 8A and 8B show a multi-channel bioresorbable nerve regeneration conduit obtaining by placing the multi-channel filler, which is wound into a spiral shape, into the hollow round tube of FIG. 7. The nerve regeneration conduit of the present invention preferably has a plurality of channels, most preferably more than 10 channels.

According to the present invention, the bioresorbable polymer material suitable for the porous bioresorbable film with an uneven surface can be polycaprolactone (PCL), polylactic acid (PLA), polyglycolic acid (PGA), poly-lactic-co-glycolic acid copolymer (PLGA copolymer), polycaprolactone-polylactic acid copolymer (PCL-PLA copolymer), polycaprolactone-polyglycolic acid copolymer (PCL-PGA copolymer), polycaprolactone-polyethylene glycol copolymer (PCL-PEG copolymer), or mixtures thereof. The bioresorbable polymer can have a molecular weight higher than 20,000, and preferably 20,000 to 300,000.

The bioresorbable polymer material suitable for the hollow round tube can be polycaprolactone (PCL), polylactic acid (PLA), polyglycolic acid (PGA), poly-lactic-co-glycolic acid copolymer (PLGA copolymer), polycaprolactone-polylactic acid copolymer (PCL-PLA copolymer), polycaprolactone-polyglycolic acid copolymer (PCL-PGA copolymer), polycaprolactone-polyethylene glycol copolymer (PCL-PEG copolymer), or mixtures thereof. The bioresorbable polymer can have a molecular weight higher than 20,000, and preferably 20,000 to 300,000.

According to the present invention, during the procedure of forming the multi-channel filler with an uneven surface and that of forming the hollow round tube, a low molecular weight oligomer can be added into the bioresorbable polymer solution, serving as a pore former.

Specifically speaking, during the procedure of forming the multi-channel filler, a bioresorbable polymer and a low molecular weight oligomer are dissolved together in an organic solvent to form a bioresorbable polymer solution.

Next, according to the above-mentioned same procedures, the bioresorbable polymer solution is made to have a film shape with an uneven surface, contacted with a coagulant to form a porous bioresorbable film with an uneven surface, and finally wound into a spiral shape, forming a multi-channel filler.

During the procedure of forming the hollow round tube, a bioresorbable polymer and a low molecular weight oligomer are dissolved together in an organic solvent to form a bioresorbable polymer solution. Next, according to the above-mentioned same procedures, the bioresorbable polymer solution is made to have a hollow round tube shape, and then contacted with a coagulant to form a porous bioresorbable hollow round tube.

The low molecular weight oligomer suitable for use in the present invention can have a molecular weight of 200 to 4000. Representative examples include polycaprolactone triol (PCLTL), polycaprolactone diol (PCLDL), polycaprolactone (PCL), polylactic acid (PLA), polyethylene glycol (PEG), polypropylene glycol (PPG), polytetramethylene glycol (PTMG), and mixtures thereof.

Since the low molecular weight oligomer has considerable molecular weight, it diffuses into the coagulant at a slower rate in the precipiation process of the bioresorbable polymer solution. In this manner, a porous bioresorbable material having uniform interconnected pores is formed. Therefore, the low molecular weight oligomer acts as a pore former in the present invention. The porosity and pore size of the finally-formed hollow round tube and the multi-channel filler in the tube can be adjusted by means of choosing the species and molecular weight of the low molecular weight oligomer and the content in the bioresorbable polymer solution. In addition, both of the hollow round tube and the multi-channel filler in it become an interconnected form.

According to the present invention, the organic solvent for dissolving the bioresorbable polymer and low molecular weight oligomer can be N,N-dimethylformamide (DMF), N,N-dimethylacetamide (DMAc), THF, alcohols, chloroform, 1,4-dioxane, or mixtures thereof. The bioresorbable polymer can be present in an amount of 5-50%, more preferably 10-40%, weight fraction of the bioresorbable polymer solution. The low molecular weight oligomer can be present in an amount of 10-80% weight fraction based on the non-solvent portion of the bioresorbable polymer solution.

According to the present invention, the above coagulant preferably includes water and an organic solvent. The organic solvent in the coagulant can be present in an amount of 10-50% weight fraction. The organic solvent in the coagulant can be amides, ketones, alcohols, or mixtures thereof. Preferably, the organic solvent in the coagulant includes a ketone and an alcohol.

Representative examples of the organic solvent in the coagulant include N,N-dimethylformamide (DMF), N,N-dimethylacetamide (DMAc), ketones such as acetone and methyl ethyl ketone (MEK), and alcohols such as methanol, ethanol, propanol, isopropanol, and butanol.

After the bioresorbable polymer solution contacts the coagulant, the obtained porous bioresorbable material is preferably placed in a washing liquid for washing. The washing liquid can include water and an organic solvent such as ketones, alcohols, or mixtures thereof. Representative examples of the ketone include acetone and methyl ethyl ketone (MEK). Representative examples of the alcohol include methanol, ethanol, propanol, isopropanol and butanol.

The following examples are intended to illustrate the process and the advantages of the present invention more fully without limiting its scope, since numerous modifications and variations will be apparent to those skilled in the art.

### Preparation of Porous Film Pre-form of Bioresorbable Polymer

### EXAMPLE (A1)

15 g of polycaprolactone (PCL) having a molecular weight about 80,000 and 15 g of polyethylene glycol (PEG) having a molecular weight of 300 (an oligomer) were added to 70 g of THF, which was stirred thoroughly at room temperature to form a PCL solution containing PEG oligomer. The solution was then coated or poured onto the surface of a mold with an uneven (textured) surface.

The mold coated with PCL solution was then placed in a coagulant at 25° C. (the composition of the coagulant and coagulating time are shown in Table 1). Thus, the PCL solution was coagulated to form a porous PCL material. The porous PCL material was then immersed in a 50 wt % ethanol solution (washing liquid) for 2 hours, and then washed with clean water and dried to obtain the final porous PCL pre-form material with an uneven surface (Nos. #1A-#1K). The base of the pre-form material obtained had a thickness of about 0.1 mm, and the protruding depth was about 0.2 mm.

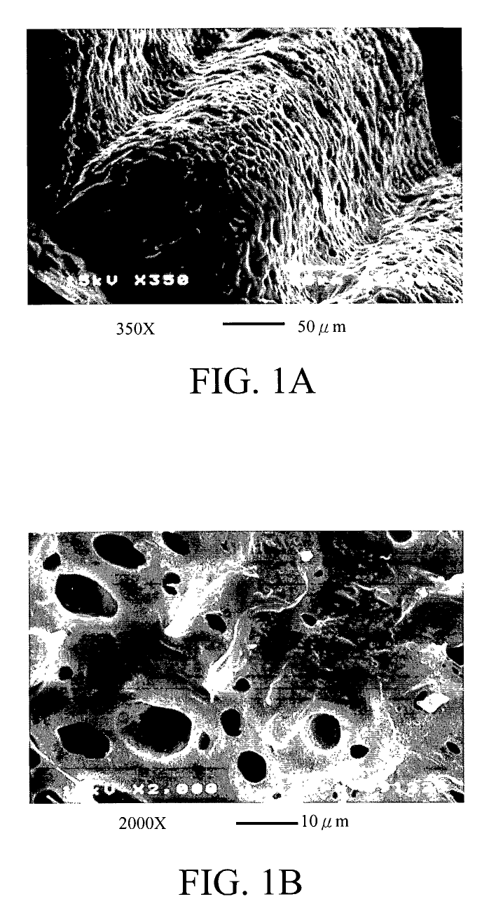

Specimens were observed by SEM (scanning electron microscope) as shown in FIGS. 1A to 1F to assure that the porous PCL pre-form material had an interconnected pore structure.

TABLE 1

| | | | Porous structure | |
|---|---|---|---|---|
| | | Coagulating | and appearance of | SEM |
| Specimen | Coagulant | time (hr) | porous matrix | photo |
| 1A | 30 wt % | 4 | interconnected | |
| | ethanol | | pores, concave and | |
| | | | convex surface | |
| 1B | 40 wt % | 4 | interconnected | |
| | ethanol | | pores, concave and | (350X) |
| | | | convex surface | |
| 1C | 45 wt % | 4 | interconnected | |
| | ethanol | | pores, concave and | |
| | | | convex surface | |
| 1D | 50 wt % | 4 | interconnected | |
| | ethanol | | pores, concave and | |
| | | | convex surface | |
| 1E | 30 wt % | 4 | interconnected | |
| | acetone | | pores, concave and | (2000X) |
| | | | convex surface | |
| 1F | 40 wt % | 4 | interconnected | |
| | acetone | | pores, concave and | (100X) |
| | | | convex surface | |
| 1G | 45 wt % | 4 | interconnected | |
| | acetone | | pores, concave and | |
| | | | convex surface | |
| 1H | 50 wt % | 4 | interconnected | |
| | acetone | | pores, concave and | (350X) |
| | | | convex surface | |
| 1I | 15 wt % | 4 | interconnected | |
| | acetone + | | pores, concave and | (500X) |
| | 15% ethanol | | convex surface | |
| 1J | 20 wt % | | interconnected | |
| | acetone + | | pores, concave and | (350X) |
| | 20% ethanol | | convex surface | |
| 1K | 25 wt % | 4 | interconnected | |
| | acetone + | | pores concave and | |
| | 25% ethanol | | convex surface | |

### EXAMPLE (A2)

15 g of polycaprolactone (PCL) having a molecular weight about 80,000 and 15 g of PCLTL (polycaprolactone triol) having a molecular weight of 300 (an oligomer) were added to 70 g of THF, which was stirred thoroughly at room temperature to form a PCL solution containing PCLTL oligomer. The solution was then coated or poured onto the surface of a mold with an uneven (textured) surface.

The mold coated with PCL solution was then placed in a coagulant at 25° C. (the composition of the coagulant and coagulating time are shown in Table 2). Thus, the PCL solution was coagulated to form a porous PCL material. The porous PCL material was then immersed in a 50 wt % ethanol solution (washing liquid) for 2 hours, and then washed with clean water and dried to obtain the final porous PCL pre-form material with an uneven surface (Nos. #2A-#2B).

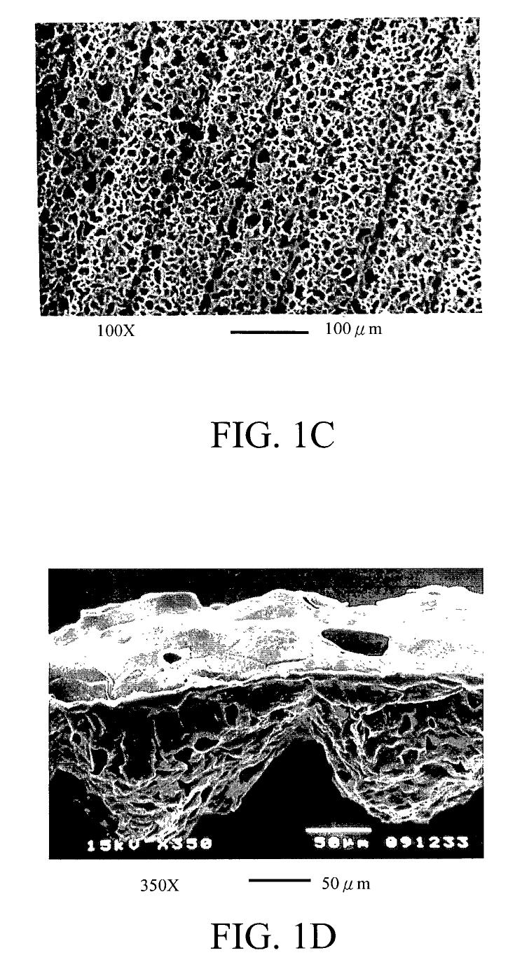

Specimen #2B was observed by SEM to assure that the porous PCL pre-form material obtained had an interconnected pore structure. The results are shown in Table 2 and SEM photograph is shown in FIG. 2.

TABLE 2

| | | | Porous structure | |
|---|---|---|---|---|
| | | Coagulating | and appearance of | |
| Specimen | Coagulant | time (hr) | porous matrix | SEM photo |
| 2A | 40 wt % | 4 | interconnected | |
| | ethanol | | pores, concave and | (1000X) |
| | | | convex surface | |
| 2B | 40 wt % | 4 | interconnected | |
| | acetone | | pores, concave and | |
| | | | convex surface | |

### EXAMPLE (A3)

15 g of polycaprolactone (PCL) having a molecular weight about 80,000 and 15 g of PTMG (polytetramethylene glycol) having a molecular weight of 1000 (an oligomer) were added to 70 g of THF, which was stirred thoroughly at room temperature to form a PCL solution containing PTMG oligomer.

The solution was then coated or poured onto the surface of a mold with an uneven (textured) surface. The mold coated with PCL solution was then placed in a coagulant at 25° C. (the composition of the coagulant and coagulating time are shown in Table 3). Thus, the PCL solution was coagulated to form a porous PCL material. The porous PCL material was then immersed in a 50 wt % ethanol solution (washing liquid) for 2 hours, and then washed with clean water and dried to obtain the final porous PCL pre-form material with an uneven surface (Nos. #3A-#3B).

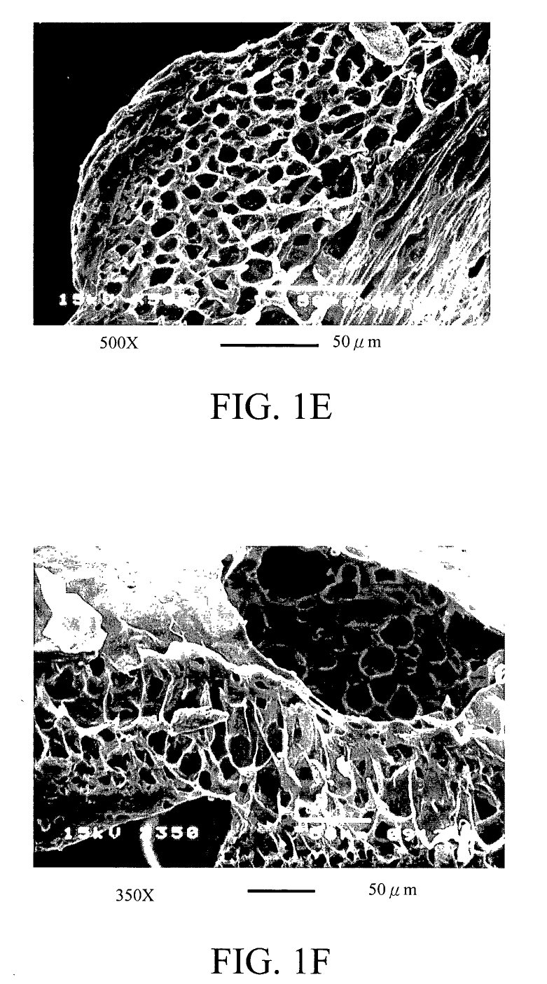

Specimen #3B was observed by SEM to assure that the porous PCL pre-form material obtained had an interconnected pore structure. The results are shown in Table 3 and SEM photograph is shown in FIG. 3.

TABLE 3

| | | | Porous structure | |
|---|---|---|---|---|
| | | Coagulating | and appearance of | |
| Specimen | Coagulant | time (hr) | porous matrix | SEM photo |
| 3A | 40 wt % | 4 | interconnected | |
| | ethanol | | pores, concave and | |
| | | | convex surface | |
| 3B | 40 wt % | 4 | interconnected | |
| | acetone | | pores, concave and | (3500X) |
| | | | convex surface | |

### EXAMPLE (A4)

15 g of polycaprolactone (PCL) having a molecular weight about 80,000 and 15 g of PEG (polyethylene glycol) having a molecular weight of 300 (an oligomer) were added to 70 g of THF, which was stirred thoroughly at room temperature to form a PCL solution containing PEG oligomer. The solution was then coated or poured onto the surface of a mold with an uneven (textured) surface, i.e., with a plurality of trenches. The depth of the trench is shown in Table 4. The trench depth determines the protrusion depth of the porous PCL pre-form to be formed in the future.

The mold coated with PCL solution was then placed in a coagulant at 25° C. (the composition is 40/60 wt % ethanol/water). Thus, the PCL solution was coagulated to form a porous PCL material. The porous PCL material was then immersed in a 50 wt % ethanol solution (washing liquid) for 2 hours, and then washed with clean water and dried to obtain the final porous PCL pre-form material with an uneven surface (Nos. #4A, #4B, and #4C).

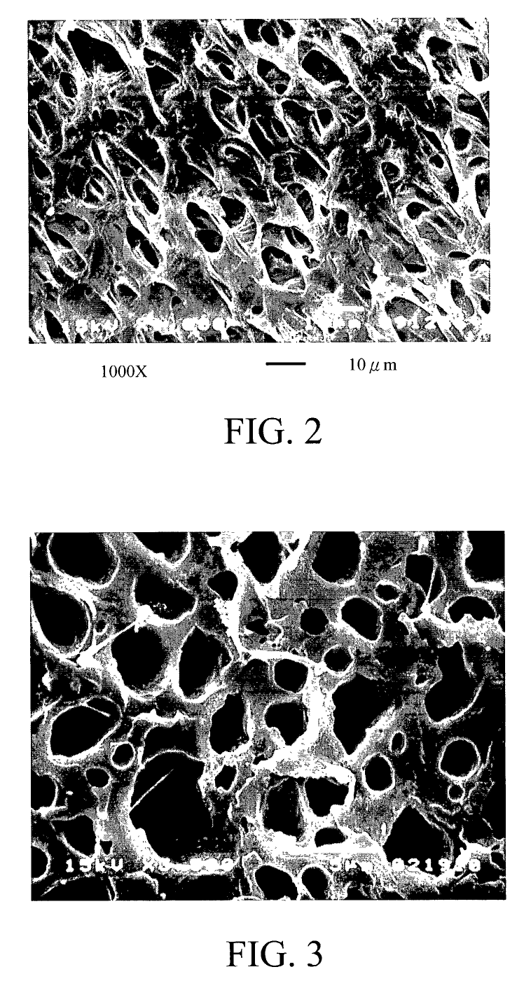

Specimens were observed by SEM as shown in FIGS. 4A and 4B to assure that the porous PCL pre-form material obtained had an interconnected pore structure and a concave and convex surface. The results are shown in Table 4.

TABLE 4

| | Trench | | Porous structure | |
|---|---|---|---|---|
| | depth of | Coagulating | and appearance of | |
| Specimen | the mold | time (hr) | porous matrix | SEM photo |
| 4A | 0.1 mm | 4 | interconnected | |
| | | | pores, concave and | (500X) |
| | | | convex surface | |
| 4B | 0.2 mm | 4 | interconnected | |
| | | | pores, concave and | (350X) |
| | | | convex surface | |
| 4C | 0.3 mm | 4 | interconnected | |
| | | | pores, concave and | |
| | | | convex surface | |

### Preparation of Porous Hollow Round Tube of Bioresorbable Polymer

### EXAMPLE (B1)

15 g of polycaprolactone (PCL) having a molecular weight about 80,000 and 15 g of PEG (polyethylene glycol) having a molecular weight of 300 (an oligomer) were added to 70 g of THF, which was stirred thoroughly at room temperature to form a PCL solution containing PEG oligomer. The solution was then poured into a cylinder-shaped coater having a round center hole with a diameter of 3.0 mm. Next, a rod with an outer diameter of 2 mm was passed through the round center hole of the coater. Thus, a PCL homogeneous solution with a thickness of 0.5 mm was coated on the rod.

The rod coated with PCL solution was then placed in a coagulant at 25° C. (the composition of the coagulant and coagulating time are shown in Table 5). Thus, the PCL solution was coagulated to form a porous PCL material in the form of a round tube. Then, the porous PCL round tube was drawn from the rod, immersed in a 50 wt % acetone solution (washing liquid) for 2 hours, washed with clean water, and dried to obtain the final porous PCL hollow round tube (Nos. #5A-#5B).

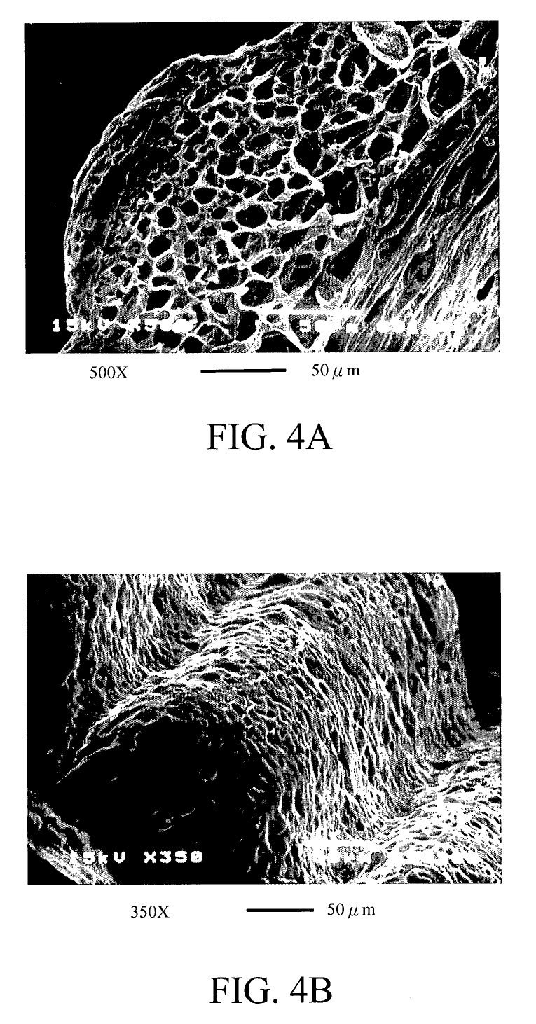

Specimens were observed by SEM as shown in FIGS. 5A and 5B to assure that the porous PCL hollow round tube obtained had an interconnected pore structure. The results are shown in Table 5.

TABLE 5

| | | | Porous structure | |
|---|---|---|---|---|
| | | Coagulating | and appearance of | |
| Specimen | Coagulant | time (hr) | porous matrix | SEM photo |
| 5A | 40 wt % | 4 | interconnected | |
| | ethanol | | pores, concave and | (200X) |
| | | | convex surface | |
| 5B | 40 wt % | 4 | interconnected | |
| | acetone | | pores, concave and | (750X) |
| | | | convex surface | |

### EXAMPLE (B2)

15 g of polycaprolactone (PCL) having a molecular weight about 80,000 and 15 g of PCLTL (polycaprolactone triol) having a molecular weight of 300 (an oligomer) were added to 70 g of THF, which was stirred thoroughly at room temperature to form a PCL solution containing PCLTL oligomer. The solution was then poured into a cylinder-shaped coater having a round center hole with a diameter of 3.0 mm. Next, a rod with an outer diameter of 2 mm was passed through the round center hole of the coater. Thus, a PCL homogeneous solution with a thickness of about 0.5 mm was coated on the rod.

The rod coated with PCL solution was then placed in a coagulant at 25° C. (the composition of the coagulant and coagulating time are shown in Table 6). Thus, the PCL solution was coagulated to form a porous PCL material in the form of a round tube. Then, the porous PCL round tube was drawn from the rod, immersed in a 50 wt % ethanol solution (washing liquid) for 2 hours, washed with clean water, and dried to obtain the final porous PCL hollow round tube (Nos. #6A-#6B).

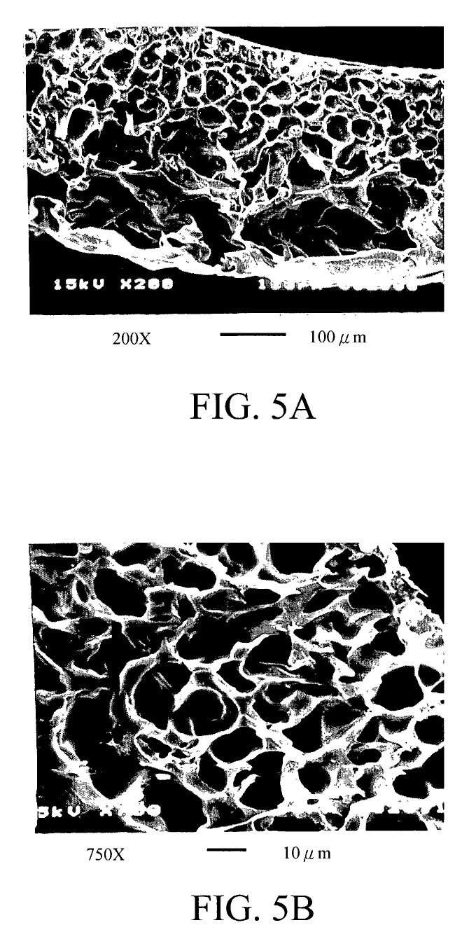

Specimen #6B was observed by SEM as shown in FIG. 6 to assure that the porous PCL hollow round tube obtained had an interconnected pore structure. The results are shown in Table 6.

TABLE 6

| | | | Porous structure | |
|---|---|---|---|---|
| | | Coagulating | and appearance of | |
| Specimen | Coagulant | time (hr) | porous matrix | SEM photo |
| 6A | 40 wt % | 4 | interconnected | |
| | ethanol | | pores, concave and | |
| | | | convex surface | |
| 6B | 40 wt % | 4 | interconnected | |
| | acetone | | pores, concave and | (200X) |
| | | | convex surface | |

### EXAMPLE (B3)

15 g of polycaprolactone (PCL) having a molecular weight about 80,000 and 15 g of PEG (polyethylene glycol) having a molecular weight of 300 (an oligomer) were added to 70 g of THF, which was stirred thoroughly at room temperature to form a PCL solution containing PEG oligomer. The solution was then poured into a cylinder-shaped coater having a round center hole with a diameter of 3.0 to 6.0 mm. Next, a rod with an outer diameter of 2.0 to 4.0 mm was passed through the round center hole of the coater. The size of the cylinder-shape coater is shown in Table 7. Thus, a PCL homogeneous solution with a thickness of 0.5 to 1.0 mm was coated on the rod.

The rod coated with PCL solution was then placed in a coagulant at 25° C. (the composition was 40/60 wt % ethanol/water). Thus, the PCL solution was coagulated to form a porous PCL material in the form of a round tube. Then, the porous PCL round tube was drawn from the rod, immersed in a 50 wt % ethanol solution (washing liquid) for 2 hours, washed with clean water, and dried to obtain the final porous PCL hollow round tube (Nos. #7A-#7C).

Specimen #7A was observed by SEM as shown in FIG. 7 to assure that the porous PCL hollow round tube obtained had an interconnected pore structure. The results are shown in Table 7.

TABLE 7

| | Size of the coater | | Porous | |
|---|---|---|---|---|
| | (round center | | structure and | |
| | hole/rod) | Coagulating | appearance of | SEM |
| Specimen | (unit: mm) | time (hr) | porous matrix | photo |
| 7A | 3.0/2.0 | 4 | interconnected | |
| | | | pores, concave | (50X) |
| | | | and convex | |
| | | | surface | |
| 7B | 4.5/3.2 | 4 | interconnected | |
| | | | pores, concave | |
| | | | and convex | |
| | | | surface | |
| 7C | 6.0/4.0 | 4 | interconnected | |
| | | | pores, concave | |
| | | | and convex | |
| | | | surface | |

### Multi-Channel Bioresorbable Nerve Conduit

### EXAMPLE (C1)

The porous bioresorbable PCL film pre-forms with an uneven surface (concave and convex surface) obtained from Example (A1) to (A4) were wound into a spiral shaped round tube respectively. The spiral shaped round tube was then placed into the hollow round tube obtained from Examples (B1) to (B3). The size of the hollow round tube was shown in Table 8. Thus, multi-channel bioresorbable nerve regeneration conduits were formed (Nos. #8A, #8B, and #8C).

The multi-channel bioresorbable nerve regeneration conduits were observed by SEM as shown in FIGS. 8A and 8B. It can be seen that the conduit had about 150 channels and had an interconnected pore structure. The results are shown in Table 8.

TABLE 8

| | Size of hollow round | | |
|---|---|---|---|
| | tube of porous | | |
| | bioresorbable polymer | | |
| | (outer diameter/ | Porous structure | |
| | inner diameter) | and appearance of | |
| Specimen | (unit: mm) | porous matrix | SEM photo |
| 8A | 3.0/2.0 | interconnected | |
| | | pores, concave and | (50X) |
| | | convex surface | |
| 8B | 4.5/3.2 | interconnected | |
| | | pores, concave and | (35X) |
| | | convex surface | |
| 8C | 6.0/4.0 | interconnected | |
| | | pores, concave and | |
| | | convex surface | |

The foregoing description of the preferred embodiments of this invention has been presented for purposes of illustration and description. Obvious modifications or variations are possible in light of the above teaching. The embodiments chosen and described provide an excellent illustration of the principles of this invention and its practical application to thereby enable those skilled in the art to utilize the invention in various embodiments and with various modifications as are suited to the particular use contemplated. All such modifications and variations are within the scope of the present invention as determined by the appended claims when interpreted in accordance with the breadth to which they are fairly, legally, and equitably entitled.

What is claimed is:

1. A multi-channel bioresorbable nerve regeneration conduit, comprising:
a hollow round tube of a porous bioresorbable polymer; and
a multi-channel filler in the round tube, which is a porous bioresorbable polymer film with an uneven surface.

2. The nerve regeneration conduit as claimed in claim 1, wherein the hollow round tube has a wall with a thickness of 0.05 to 1.5 mm.

3. The nerve regeneration conduit as claimed in claim 1, wherein the pore on the wall of the hollow round tube is interconnected.

4. The nerve regeneration conduit as claimed in claim 1, wherein the hollow round tube is a porous bioresorbable polymer and is polycaprolactone (PCL), polylactic acid (PLA), polyglycolic acid (PGA), poly-lactic-co-glycolic acid copolymer (PLGA copolymer), polycaprolactone-polylactic acid copolymer (PCL-PLA copolymer), polycaprolactone-polyglycolic acid (PCL-PGA copolymer), polycaprolactone-polyethylene glycol copolymer (PCL-PEG copolymer), or mixtures thereof.

5. The nerve regeneration conduit as claimed in claim 1, wherein the conduit has more than 10 channels.

6. The nerve regeneration conduit as claimed in claim 1, wherein the porous bioresorbable polymer film with an uneven surface includes a base and a plurality of protrusions protruding from the surface of the base, and wherein the base has a thickness of 0.05 mm to 1.0 mm.

7. The nerve regeneration conduit as claimed in claim 6, wherein in the porous bioresorbable polymer film with an uneven surface, the protrusion has a protruding depth of 0.05 mm to 1.0 mm.

8. The nerve regeneration conduit as claimed in claim 1, wherein the porous bioresorbable polymer film with an uneven surface is polycaprolactone (PCL), polylactic acid (PLA), polyglycolic acid (PGA), poly-lactic-co-glycolic acid copolymer (PLGA copolymer), polycaprolactone-polylactic acid copolymer (PCL-PLA copolymer), polycaprolactone-polyglycolic acid (PCL-PGA copolymer), polycaprolactone-polyethylene glycol copolymer (PCL-PEG copolymer), or mixtures thereof.

9. The nerve regeneration conduit as claimed in claim 1, wherein the porous bioresorbable polymer film with an uneven surface is single layer, multiple layer, in a folded form, or wound into a spiral shape.

10. The nerve regeneration conduit as claimed in claim 9, wherein the porous bioresorbable polymer film with an uneven surface is wound into a spiral shape.

11. A process for preparing a multi-channel bioresorbable nerve regeneraton conduit, comprising:
forming a multi-channel filler, which is a porous bioresorbable polymer film with an uneven surface;
forming a hollow round tube of a porous bioresorbable polymer; and
placing the multi-channel filler into the hollow round tube.

12. The process as claimed in claim 11, wherein porous bioresorbable polymer film with an uneven surface is formed by the following steps:
dissolving a bioresorbable polymer in an organic solvent to form a bioresorbable polymer solution;
making the bioresorbable polymer solution have a film shape with an uneven surface; and
contacting the film-shaped solution with a coagulant to form a porous bioresorbable film with an uneven surface.

13. The process as claimed in claim 12, wherein the step of forming the bioresorbable polymer solution further comprises dissolving a low molecular weight oligomer in the organic solvent, wherein the low molecular weight oligomer has a molecular weight of 200 to 4000.

14. The process as claimed in claim 13, wherein the low molecular weight oligomer is polycaprolactone triol (PCLTL), polycaprolactone diol (PCLDL), polycaprolactone (PCL), polylactic acid (PLA), polyethylene glycol (PEG), polypropylene glycol (PPG), polytetramethylene glycol (PTMG), or mixtures thereof.

15. The process as claimed in claim 11, wherein the hollow round tube of porous bioresorbable polymer is formed by the following steps:
dissolving a bioresorbable polymer in an organic solvent to form a bioresorbable polymer solution;
making the bioresorbable polymer solution have a hollow round tube shape; and
contacting the hollow round tube-shaped solution with a coagulant to form the hollow round tube of the porous bioresorbable polymer.

16. The process as claimed in claim 15, wherein the hollow round tube of porous bioresorbable polymer is formed by the following steps:
dissolving a bioresorbable polymer in an organic solvent to form a bioresorbable polymer solution;
coating the bioresorbable polymer solution onto a surface of a rod to make the solution have a round tube shape;
placing the rod coated with the bioresorbable polymer solution into a coagulant to form a round tube shaped-porous bioresorbable material on the surface of the rod; and
drawing out the round tube-shaped porous bioresorbable material from the surface of the rod to obtain the porous bioresorbable hollow round tube.

17. The process as claimed in claim 16, wherein the step of forming the bioresorbable polymer solution further comprising dissolving a low molecular weight oligomer in the organic solvent, wherein the low molecular weight oligomer has a molecular weight of 200 to 4000.

18. The process as claimed in claim 17, wherein the low molecular weight oligomer is polycaprolactone triol (PCLTL), polycaprolactone diol (PCLDL), polycaprolactone (PCL), polylactic acid (PLA), polyethylene glycol (PEG), polypropylene glycol (PPG), polytetramethylene glycol (PTMG), or mixtures thereof.

19. The process as claimed in claim 11, wherein the porous bioresorbable polymer film with an uneven surface is single layer, multiple layer, in a folded form, or wound into a spiral shape.

20. The process as claimed in claim 19, wherein the porous bioresorbable polymer film with an uneven surface is wound into a spiral shape.
